(19) United States
(12) Patent Application Publication
(10) Pub. No.: US 2026/0144632 A1
(43) Pub. Date: May 28, 2026

(54) PROSTHETIC VALVE DOCKING DEVICE

(71) Applicant: Edwards Lifesciences Corporation, Irvine, CA (US)

(72) Inventors: Jocelyn Chau, Buena Park, CA (US); Tram Ngoc Nguyen, Santa Ana, CA (US)

(21) Appl. No.: 19/450,365

(22) Filed: Jan. 15, 2026

Related U.S. Application Data
(63) Continuation of application No. PCT/US2024/037020, filed on Jul. 8, 2024.
(60) Provisional application No. 63/514,549, filed on Jul. 19, 2023.

(51) Int. Cl. A61F 2/24

(52) U.S. Cl. CPC A61F 2/2418; A61F 2210/0014; A61F 2220/0008; A61F 2250/0037

(57) ABSTRACT

A docking device for securing a prosthetic valve at a native valve includes a coil having a plurality of helical turns when deployed at the native valve, and a guard member attached to the coil. The guard member includes braided wires and is movable between a radially compressed state and a radially expanded state. The guard member includes a plurality of body portions and one or more neck portions connecting the plurality of body portions. When the guard member is in the radially expanded state, a diameter of the neck portions is smaller than a diameter of the body portions.

### CROSS-REFERENCE TO RELATED APPLICATIONS

This application is a continuation of PCT Patent Application No. PCT/US2024/037020 filed on July 8, 2024, which claims the benefit of U.S. Provisional Application No. 63/514,549, filed July 19, 2023, each of these applications being incorporated by reference in its entirety herein.

### FIELD

The present disclosure concerns examples of a docking device configured to secure a prosthetic valve at a native heart valve, as well as methods of assembling such devices.

### BACKGROUND

Prosthetic valves can be used to treat cardiac valvular disorders. Native heart valves (for example, the aortic, pulmonary, tricuspid and mitral valves) function to prevent backward flow or regurgitation, while allowing forward flow. These heart valves can be rendered less effective by congenital, inflammatory, infectious conditions, etc. Such conditions can eventually lead to serious cardiovascular compromise or death. For many years, the doctors attempted to treat such disorders with surgical repair or replacement of the valve during open heart surgery.

A transcatheter technique for introducing and implanting a prosthetic heart valve using a catheter in a manner that is less invasive than open heart surgery can reduce complications associated with open heart surgery. In this technique, a prosthetic valve can be mounted in a compressed state on the end portion of a catheter and advanced through a blood vessel of the patient until the valve reaches the implantation site. The valve at the catheter tip can then be expanded to its functional size at the site of the defective native valve, such as by inflating a balloon on which the valve is mounted or, for example, the valve can have a resilient, self-expanding frame that expands the valve to its functional size when it is advanced from a delivery sheath at the distal end of the catheter. Optionally, the valve can have a balloon-expandable, self-expanding, mechanically expandable frame, and/or a frame expandable in multiple or a combination of ways.

In some instances, a transcatheter heart valve (THV) may be appropriately sized to be placed inside a particular native valve (for example, a native aortic valve). As such, the THV may not be suitable for implantation at another native valve (for example, a native mitral valve) and/or in a patient with a larger native valve. Additionally, or alternatively, the native tissue at the implantation site may not provide sufficient structure for the THV to be secured in place relative to the native tissue. Accordingly, improvements to THVs and the associated transcatheter delivery apparatus are desirable.

### SUMMARY

The present disclosure relates to methods and devices for treating valvular regurgitation and/or other valve issues. Specifically, the present disclosure is directed to a docking device configured to receive a prosthetic valve and the methods of assembling the docking device and implanting the docking device.

A docking device for securing a prosthetic valve at a native valve can include a coil comprising a plurality of helical turns when deployed at the native valve. In addition to these features, a docking device can further comprise one or more of the components disclosed herein.

In certain examples, a docking device can include a guard member attached to the coil.

In certain examples, the guard member includes braided wires and is movable between a radially compressed state and a radially expanded state.

In certain examples, the guard member includes a plurality of body portions and one or more neck portions connecting the plurality of body portions.

In certain examples, when the guard member is in the radially expanded state, a diameter of the neck portions is smaller than a diameter of the body portions.

In certain examples, when the guard member is in the radially compressed state, both the body portions and neck portions are reduced to a compressed diameter.

In certain examples, when the guard member is in the radially expanded state, the neck portions are radially expanded to a first expanded diameter that is larger than the compressed diameter, and the body portions are radially expanded to a second expanded diameter that is larger than the first expanded diameter.

Certain aspects of the disclosure concern a guard member for a docking device configured to receive a prosthetic valve. The guard member can include a braided wire frame such that the guard member is movable between a radially compressed state and a radially expanded state. The guard member can include a plurality of body portions and one or more neck portions connecting the plurality of body portions. When the guard member is in the radially compressed state, both the body portions and neck portions are reduced to a compressed diameter. When the guard member is in the radially expanded state, the neck portions are radially expanded to a first expanded diameter that is larger than the compressed diameter, and the body portions are radially expanded to a second expanded diameter that is larger than the first expanded diameter.

Certain aspects of the disclosure concern a method for implanting a prosthetic valve. The method includes deploying a docking device at an annulus of a native valve, and deploying a prosthetic valve within the docking device. The docking device includes a coil and a guard member attached to the coil, the coil having a helical configuration after being deployed. The guard member includes braided wires and is movable between a radially compressed state and a radially expanded state. When the guard member is in the radially expanded state, a diameter of the neck portions is smaller than a diameter of the body portions.

In certain examples, deploying the docking device includes moving the guard member from the radially compressed state to the radially expanded state, and aligning one of the neck portions of the guard member to a commissure of the native valve such that the commissure is positioned between two adjacent body portions of the guard member.

The above method can be performed on a living animal or on a simulation, such as on a cadaver, cadaver heart, anthropomorphic ghost, simulator (for example, with body parts, heart, tissue, etc. being simulated).

In some examples, a docking device or a guard member comprises one or more of the components recited in Examples 1-34 described in the section “Additional Examples of the Disclosed Technology” below.

The foregoing and other objects, features, and advantages of the disclosed technology will become more apparent from the following detailed description, which proceeds with reference to the accompanying figures.

### BRIEF DESCRIPTION OF THE DRAWINGS

FIG. 1 schematically illustrates a first stage in an exemplary mitral valve replacement procedure where a guide catheter and a guidewire are inserted into a vasculature of a patient and navigated through the vasculature and into a heart of the patient, towards a native mitral valve of the heart.

FIG. 2A schematically illustrates a second stage in the exemplary mitral valve replacement procedure where a docking device delivery apparatus extending through the guide catheter is used to deploy a docking device at the native mitral valve.

FIG. 2B schematically illustrates a third stage in the exemplary mitral valve replacement procedure where the docking device of FIG. 2A is fully implanted at the native mitral valve of the patient and the docking device delivery apparatus has been removed from the patient.

FIG. 3A schematically illustrates a fourth stage in the exemplary mitral valve replacement procedure where a prosthetic heart valve delivery apparatus extending through the guide catheter is deploy a prosthetic heart valve within the implanted docking device at the native mitral valve.

FIG. 3B schematically illustrates a fifth stage in the exemplary mitral valve replacement procedure where the prosthetic heart valve is fully implanted within the docking device at the native mitral valve and the prosthetic heart valve delivery apparatus has been removed from the patient.

FIG. 4 schematically illustrates a sixth stage in the exemplary mitral valve replacement procedure where the guide catheter and the guidewire have been removed from the patient.

FIG. 5 is a side perspective view of a docking device in a deployed configuration, the docking device including a helical coil and a guard member, according to one example.

FIG. 6 is a side elevation view of a portion of the guard member of FIG. 5 in a radially expanded state.

FIG. 7 is a side elevation view of a portion of the guard member of FIG. 5 in a radially compressed state.

FIG. 8A is a cross-sectional view of the docking device taken along line A-A depicted in FIG. 5, according to one example.

FIG. 8B is a cross-sectional view of the docking device taken along the same line A-A depicted in FIG. 5, except in FIG. 8B, the docking device is in a substantially straight delivery configuration.

FIG. 8C is a cross-sectional view of the docking device taken along line B-B depicted in FIG. 5, according to one example.

FIG. 8D is a cross-sectional view of the docking device taken along the same line B-B depicted in FIG. 5, except in FIG. 8D, the docking device is in a substantially straight delivery configuration.

FIG. 9 is a side elevation view of a portion of the guard member of FIG. 5 in a semi-compressed state caused by radial expansion of a prosthetic valve within the docking device.

FIG. 10 is a cross-sectional view of the docking device taken along the same line A-A as depicted in FIG. 5, except in FIG. 10, the docking device is in the semi-compressed state caused by radial expansion of a prosthetic valve within the docking device.

FIG. 11 is a cross-sectional view of the docking device taken along the same line B-B as depicted in FIG. 5, except in FIG. 10, the docking device is in the semi-compressed state caused by radial expansion of a prosthetic valve within the docking device.

FIG. 12 depicts the docking device of FIG. 5 being deployed at a native heart valve annulus.

FIG. 13 depicted a wire frame braided onto a mandrel, according to one example.

FIG. 14 depicts the wire frame of FIG. 13 on the mandrel after being shape-set.

FIG. 15 is a perspective view of an example prosthetic heart valve.

### DETAILED DESCRIPTION

### General Considerations

It should be understood that the disclosed examples can be adapted to deliver and implant prosthetic devices in any of the native annuluses of the heart (for example, the pulmonary, mitral, and tricuspid annuluses), and can be used with any of various delivery approaches (for example, retrograde, antegrade, transseptal, transventricular, transatrial, etc.).

For purposes of this description, certain aspects, advantages, and novel features of the examples of this disclosure are described herein. The disclosed methods, apparatus, and systems should not be construed as being limiting in any way. Instead, the present disclosure is directed toward all novel and nonobvious features and aspects of the various disclosed examples, alone and in various combinations and sub-combinations with one another. The methods, apparatus, and systems are not limited to any specific aspect or feature or combination thereof, nor do the disclosed examples require that any one or more specific advantages be present or problems be solved. The technologies from any example can be combined with the technologies described in any one or more of the other examples. In view of the many possible examples to which the principles of the disclosed technology may be applied, it should be recognized that the illustrated examples are only preferred examples and should not be taken as limiting the scope of the disclosed technology.

Although the operations of some of the disclosed examples are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

As used in this application and in the claims, the singular forms “a,” “an,” and “the” include the plural forms unless the context clearly dictates otherwise. Additionally, the term “includes” means “comprises.” Further, the terms “coupled” and “connected” generally mean electrically, electromagnetically, and/or physically (for example, mechanically or chemically) coupled or linked and does not exclude the presence of intermediate elements between the coupled or associated items absent specific contrary language.

As used herein, the term “proximal” refers to a position, direction, or portion of a device that is closer to the user and further away from the implantation site. As used herein, the term “distal” refers to a position, direction, or portion of a device that is further away from the user and closer to the implantation site. Thus, for example, proximal motion of a device is motion of the device away from the implantation site and toward the user (for example, out of the patient’s body), while distal motion of the device is motion of the device away from the user and toward the implantation site (for example, into the patient’s body). The terms “longitudinal” and “axial” refer to an axis extending in the proximal and distal directions, unless otherwise expressly defined.

Directions and other relative references (for example, inner, outer, upper, lower, etc.) may be used to facilitate discussion of the drawings and principles herein, but are not intended to be limiting. For example, certain terms may be used such as “inside,” “outside,”, “top,” “down,” “interior,” “exterior,” and the like. Such terms are used, where applicable, to provide some clarity of description when dealing with relative relationships, particularly with respect to the illustrated examples. Such terms are not, however, intended to imply absolute relationships, positions, and/or orientations. For example, with respect to an object, an “upper” part can become a “lower” part simply by turning the object over. Nevertheless, it is still the same part and the object remains the same. As used herein, “and/or” means “and” or “or,” as well as “and” and “or.”

### Exemplary Transcatheter Heart Valve Replacement Procedure

Described herein are various systems, apparatuses, methods, or the like, which can be used in or with delivery apparatuses to deliver a prosthetic implant (for example, a prosthetic valve, a docking device, etc.) into a patient body.

In certain examples, a delivery apparatus can be configured to deliver and implant a docking device at an implantation site, such as a native valve annulus. The docking device can be configured to more securely hold an expandable prosthetic valve implanted within the docking device, at the native valve annulus. For example, a docking device can provide or form a more circular and/or stable anchoring site, landing zone, or implantation zone at the implant site, in which a prosthetic valve can be expanded or otherwise implanted. By providing such anchoring or docking devices, replacement prosthetic valves can be more securely implanted and held at various valve annuluses, including at the mitral annulus which does not have a naturally circular cross-section.

In some examples, the docking device can be arranged within an outer shaft of the delivery apparatus. A sleeve shaft can cover or surround the docking device within the delivery apparatus and during delivery to a target implantation site. A pusher shaft can be disposed within the outer shaft, proximal to the docking device, and configured to push the docking device out of the outer shaft to position the docking device at the target implantation site. The sleeve shaft can also surround the pusher shaft within the outer shaft of the delivery apparatus. After positioning the docking device at the target implantation site, the sleeve shaft can be removed from the docking device and retracted back into the outer shaft of the delivery apparatus.

Fluid (for example, a flush fluid, such as heparinized saline or the like) can be provided to a pusher shaft lumen defined within an interior of the pusher shaft, a delivery shaft lumen defined between the sleeve shaft and the outer shaft of the delivery apparatus, and a sleeve shaft lumen defined between the pusher shaft and the sleeve shaft. By providing a consistent flow of fluid through these lumens of the delivery apparatus, stagnation of blood within the delivery apparatus can be reduced or avoided, thereby reducing a risk of thrombus formation.

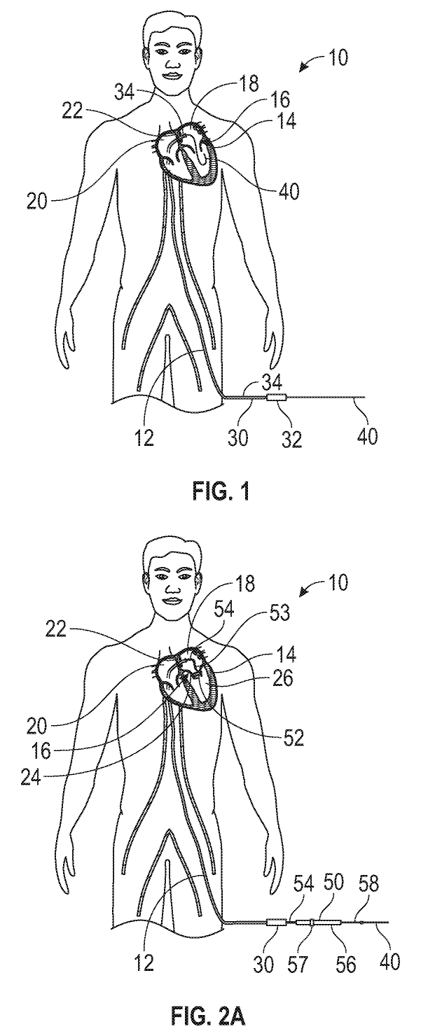

An exemplary transcatheter heart valve replacement procedure which utilizes a first delivery apparatus to deliver a docking device to a native valve annulus and then a second delivery apparatus to deliver a prosthetic heart valve (for example, THV) inside the docking device is depicted in the schematic illustrations of FIGS. 1-4.

As introduced above, defective native heart valves may be replaced with THVs. However, in certain instances, such THVs may not be able to sufficiently secure themselves to the native tissue (for example, to the leaflets and/or annulus of the native heart valve) and may undesirably shift around relative to the native tissue, leading to paravalvular leakage, valve malfunction, and/or other issues. Thus, a docking device may be implanted first at the native valve annulus and then the THV can be implanted within the docking device to help anchor the THV to the native tissue and provide a seal between the native tissue and the THV.

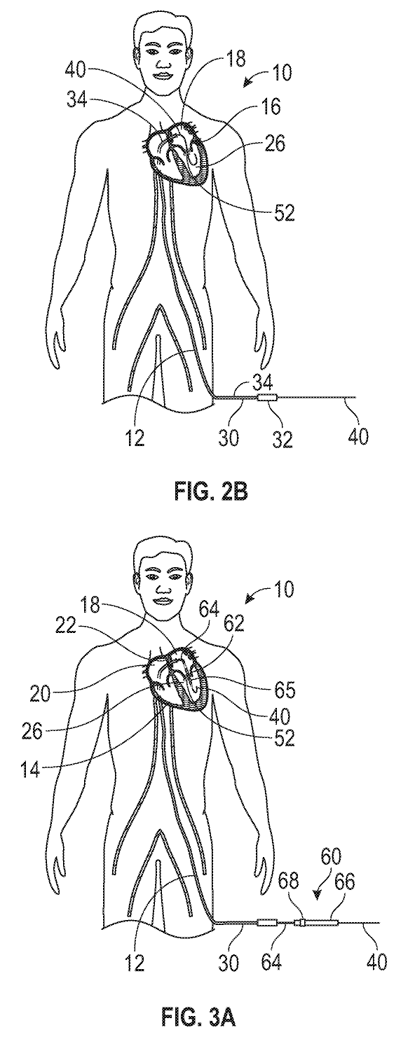

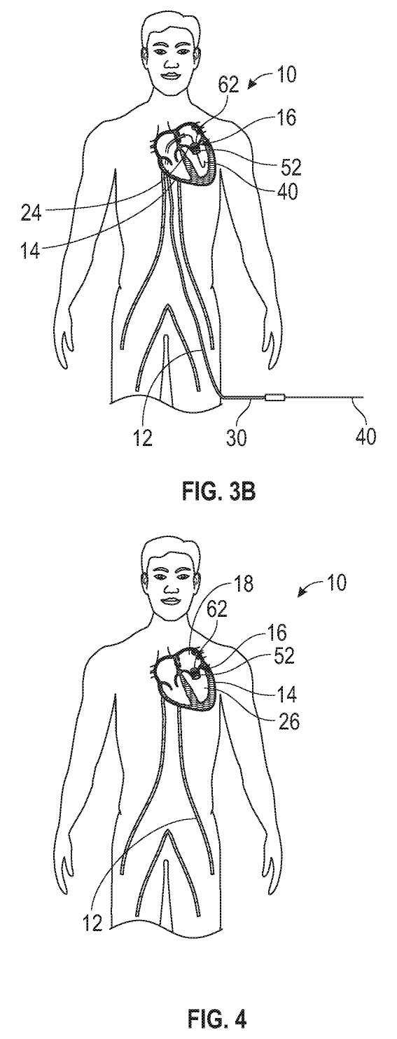

FIGS. 1-4 depict an exemplary transcatheter heart valve replacement procedure (for example, a mitral valve replacement procedure) which utilizes a docking device 52 and a prosthetic heart valve 62, according to one example. During the procedure, a user can create a pathway to a patient’s native heart valve using a guide catheter 30 (FIG. 1). The user can deliver and implant the docking device 52 at the patient’s native heart valve using a docking device delivery apparatus 50 (FIG. 2A) and then removes the docking device delivery apparatus 50 from the patient 10 after implanting the docking device 52 (FIG. 2B). The user can then implant the prosthetic heart valve 62 within the implanted docking device 52 using a prosthetic valve delivery apparatus 60 (FIG. 3A). Thereafter, the user can remove the prosthetic valve delivery apparatus 60 from the patient 10 (FIG. 3B), as well as the guide catheter 30 (FIG. 4).

FIG. 1 depicts a first stage in a mitral valve replacement procedure, according to one example. As shown, the guide catheter 30 and a guidewire 40 can be inserted into a vasculature 12 of a patient 10 and navigated through the vasculature 12, into a heart 14 of the patient 10, and toward the native mitral valve 16. Together, the guide catheter 30 and the guidewire 40 can provide a path for the docking device delivery apparatus 50 and the prosthetic valve delivery apparatus 60 to be navigated through and along, to the implantation site (for example, the native mitral valve 16 or native mitral valve annulus).

Initially, the user may first make an incision in the patient’s body to access the vasculature 12. For example, as illustrated in FIG. 1, the user may make an incision in the patient’s groin to access a femoral vein. Thus, in such examples, the vasculature 12 may include a femoral vein.

After making the incision to access the vasculature 12, the user may insert the guide catheter 30, the guidewire 40, and/or additional devices (such as an introducer device or transseptal puncture device) through the incision and into the vasculature 12. The guide catheter 30 (which can also be referred to as an “introducer device,” “introducer,” or “guide sheath”) can be configured to facilitate the percutaneous introduction of various implant delivery devices (for example, the docking device delivery apparatus 50 and the prosthetic valve delivery apparatus 60) into and through the vasculature 12 and may extend through the vasculature 12 and into the heart 14 but may stop short of the native mitral valve 16. The guide catheter 30 can comprise a handle 32 and a shaft 34 extending distally from the handle 32. The shaft 34 can extend through the vasculature 12 and into the heart 14 while the handle 32 can remain outside the body of the patient 10 and can be operated by the user in order to manipulate the shaft 34 (FIG. 1).

The guidewire 40 can be configured to guide the delivery apparatuses (for example, the guide catheter 30, the docking device delivery apparatus 50, the prosthetic valve delivery apparatus 60, additional catheters, or the like) and their associated devices (for example, docking device, prosthetic heart valve, and the like) to the implantation site within the heart 14, and thus may extend all the way through the vasculature 12 and into a left atrium 18 of the heart 14 (and in some examples, through the native mitral valve 16 and into a left ventricle of the heart 14) (FIG. 1).

In some instances, a transseptal puncture device or catheter can be used to initially access the left atrium 18, prior to inserting the guidewire 40 and the guide catheter 30. For example, after making the incision to access the vasculature 12, the user may insert a transseptal puncture device through the incision and into the vasculature 12. The user may guide the transseptal puncture device through the vasculature 12 and into the heart 14 (for example, through the femoral vein and into the right atrium 20). The user can then make a small incision in an atrial septum 22 of the heart 14 to allow access to the left atrium 18 from the right atrium 20. The user can then insert and advance the guidewire 40 through the transseptal puncture device within the vasculature 12 and through the incision in the atrial septum 22 into the left atrium 18. Once the guidewire 40 is positioned within the left atrium 18 and/or the left ventricle 26, the transseptal puncture device can be removed from the patient 10. The user can then insert the guide catheter 30 into the vasculature 12 and advance the guide catheter 30 into the left atrium 18 over the guidewire 40 (FIG. 1).

In some instances, an introducer device can be inserted through a lumen of the guide catheter 30 prior to inserting the guide catheter 30 into the vasculature 12. In some instances, the introducer device can include a tapered end that extends out a distal tip of the guide catheter 30 and that is configured to guide the guide catheter 30 into the left atrium 18 over the guidewire 40. Additionally, in some instances the introducer device can include a proximal end portion that extends out a proximal end of the guide catheter 30. Once the guide catheter 30 reaches the left atrium 18, the user can remove the introducer device from inside the guide catheter 30 and the patient 10. Thus, only the guide catheter 30 and the guidewire 40 remain inside the patient 10. The guide catheter 30 is then in position to receive an implant delivery apparatus and help guide it to the left atrium 18, as described further below.

FIG. 2A depicts a second stage in the exemplary mitral valve replacement procedure where a docking device 52 can be implanted at the native mitral valve 16 of the heart 14 of the patient 10 using a docking device delivery apparatus 50 (which may also be referred to as an “implant catheter,” or a “docking device delivery device,” or simply “delivery apparatus”).

In general, the docking device delivery apparatus 50 can include a delivery shaft 54 (which may also be referred to as an “outer shaft”), a handle 56, and a pusher assembly 58 (which may also be referred to as a “pusher shaft”). The delivery shaft 54 can be configured to be advanced through the patient’s vasculature 12 and to the implantation site (for example, native mitral valve 16) by the user, and may be configured to retain the docking device 52 in a distal end portion 53 of the delivery shaft 54. In some examples, the distal end portion 53 of the delivery shaft 54 can retain the docking device 52 therein in a substantially straightened delivery configuration.

The handle 56 of the docking device delivery apparatus 50 can be configured to be gripped and/or otherwise held by the user to advance the delivery shaft 54 through the patient’s vasculature 12. Specifically, the handle 56 can be coupled to a proximal end of the delivery shaft 54 and can be configured to remain accessible to the user (for example, outside the body of the patient 10) during the docking device implantation procedure. In this way, the user can advance the delivery shaft 54 through the patient’s vasculature 12 by exerting a force on (for example, pushing) the handle 56. In some examples, the delivery shaft 54 can be configured to carry the pusher assembly 58 and/or the docking device 52 with it as it advances through the patient’s vasculature 12. In this way, the docking device 52 and/or the pusher assembly 58 can advance through the patient’s vasculature 12 in lockstep with the delivery shaft 54 as the user grips the handle 56 and pushes the delivery shaft 54 deeper into the patient’s vasculature 12.

In some examples, the handle 56 can comprise one or more articulation members 57 that are configured to aid in navigating the delivery shaft 54 through the vasculature 12. For example, the one or more articulation members 57 can comprise one or more of knobs, buttons, wheels, and/or other types of physically adjustable control members that are configured to be adjusted by the user to flex, bend, twist, turn, and/or otherwise articulate a distal end portion 53 of the delivery shaft 54 to aid in navigating the delivery shaft 54 through the vasculature 12 and/or within the heart 14.

The pusher assembly 58 can be configured to deploy and/or implant the docking device 52 at the implantation site (for example, the native mitral valve 16). For example, the pusher assembly 58 can be configured to be adjusted by the user to push the docking device 52 out of the distal end portion 53 of the delivery shaft 54. A pusher shaft of the pusher assembly 58 can extend through the delivery shaft 54 and can be disposed adjacent to the docking device 52 within the delivery shaft 54. In some examples, the docking device 52 can be releasably coupled to the pusher shaft of the pusher assembly 58 via a connection mechanism of the docking device delivery apparatus 50 such that the docking device 52 can be released after being deployed at the native mitral valve 16. Because the docking device 52 is retained by, held, and/or otherwise coupled to the pusher assembly 58, the docking device 52 can advance in lockstep with the pusher assembly 58 through and/or out of the delivery shaft 54.

In addition to the pusher shaft, in certain instances, the pusher assembly 58 can also include a sleeve shaft. The pusher shaft can be configured to advance the docking device 52 through the delivery shaft 54 and out of the distal end portion 53 of the delivery shaft 54, while the sleeve shaft, when included, can have a distal dock sleeve configured to cover the docking device 52 within the delivery shaft 54 and while pushing the docking device 52 out of the delivery shaft 54 and positioning the docking device 52 at the implantation site. In some examples, the pusher shaft can be covered, at least in part, by the sleeve shaft.

In some examples, the pusher assembly 58 can comprise a pusher handle that is coupled to the pusher shaft and that is configured to be gripped and pushed by the user to translate the pusher shaft axially relative to the delivery shaft 54 (for example, to push the pusher shaft into and/or out of the distal end portion 53 of the delivery shaft 54). The dock sleeve can be configured to be retracted and/or withdrawn from the docking device 52, after positioning the docking device 52 at the target implantation site. For example, the pusher assembly 58 can include a sleeve handle that is coupled to the sleeve shaft and is configured to be pulled by a user to retract (for example, axially move) the sleeve shaft relative to the pusher shaft, thereby retracting the dock sleeve.

The pusher assembly 58 can be removably coupled to the docking device 52, and as such can be configured to release, detach, decouple, and/or otherwise disconnect from the docking device 52 once the docking device 52 has been deployed at the target implantation site. As just one example, the pusher assembly 58 may be removably coupled to the docking device 52 via a thread, string, yarn, suture, or other suitable material that is tied or sutured to the docking device 52.

In some examples, the pusher assembly 58 can include a suture lock assembly (also referred to as a “suture lock”) that is configured to receive and/or hold the thread or other suitable material that is coupled to the docking device 52 via a suture. The thread or other suitable material that forms the suture can extend from the docking device 52, through the pusher assembly 58, to the suture lock assembly. The suture lock assembly can also be configured to cut the suture to release, detach, decouple, and/or otherwise disconnect the docking device 52 from the pusher assembly 58. For example, the suture lock assembly can comprise a cutting mechanism that is configured to be adjusted by the user to cut the suture.

Referring again to FIG. 2A, after the guide catheter 30 is positioned within the left atrium 18, the user may insert the docking device delivery apparatus 50 (for example, the delivery shaft 54) into the patient 10 by advancing the delivery shaft 54 of the docking device delivery apparatus 50 through the guide catheter 30 and over the guidewire 40. In some examples, the guidewire 40 can be at least partially retracted away from the left atrium 18 and into the guide catheter 30. The user may then continue to advance the delivery shaft 54 of the docking device delivery apparatus 50 through the vasculature 12 along the guidewire 40 until the delivery shaft 54 reaches the left atrium 18, as illustrated in FIG. 2A. Specifically, the user may advance the delivery shaft 54 of the docking device delivery apparatus 50 by gripping and exerting a force on (for example, pushing) the handle 56 of the docking device delivery apparatus 50 toward the patient 10. While advancing the delivery shaft 54 through the vasculature 12 and the heart 14, the user may adjust the one or more articulation members 57 of the handle 56 to navigate the various turns, corners, constrictions, and/or other obstacles in the vasculature 12 and the heart 14.

Once the delivery shaft 54 reaches the left atrium 18 and extends out of a distal end of the guide catheter 30, the user can position the distal end portion 53 of the delivery shaft 54 at and/or near the posteromedial commissure of the native mitral valve 16 using the handle 56 (for example, the articulation members 57). The user may then push the docking device 52 out of the distal end portion 53 of the delivery shaft 54 with the shaft of the pusher assembly 58 to deploy and/or implant the docking device 52 within the annulus of the native mitral valve 16.

In some examples, the docking device 52 may be constructed from, formed of, and/or comprise a shape memory material, and as such, may return to its original, pre-formed shape when it exits the delivery shaft 54 and is no longer constrained by the delivery shaft 54. As one example, the docking device 52 may originally be formed as a coil, and thus may wrap around leaflets 24 of the native mitral valve 16 as it exits the delivery shaft 54 and returns to its original coiled configuration.

After pushing a ventricular portion of the docking device 52 (for example, the portion of the docking device 52 shown in FIG. 2A that is configured to be positioned within a left ventricle 26 and/or on the ventricular side of the native mitral valve 16), the user may then deploy the remaining portion of the docking device 52 (for example, an atrial portion of the docking device 52) from the delivery shaft 54 within the left atrium 18 by retracting the delivery shaft 54 away from the medial commissure of the native mitral valve 16. For example, the user can maintain the position of the pusher assembly 58 (for example, by exerting a holding and/or pushing force on the pusher shaft) while retracting the delivery shaft 54 proximally so that the delivery shaft 54 withdraws and/or otherwise retracts relative to the docking device 52 and the pusher assembly 58. In this way, the pusher assembly 58 can hold the docking device 52 in place while the user retracts the delivery shaft 54, thereby releasing the docking device 52 from the delivery shaft 54. In some examples, the user can also remove the dock sleeve from the docking device 52, for example, by retracting the sleeve shaft.

After deploying and implanting the docking device 52 at the native mitral valve 16, the user may disconnect the docking device delivery apparatus 50 from the docking device 52. Once the docking device 52 can be disconnected from the docking device delivery apparatus 50 (for example, by cutting the suture tied to the docking device 52), the user may retract the docking device delivery apparatus 50 out of the vasculature 12 and away from the patient 10 so that the user can deliver and implant a prosthetic heart valve 62 within the implanted docking device 52 at the native mitral valve 16.

FIG. 2B depicts a third stage in the mitral valve replacement procedure, where the docking device 52 has been fully deployed and implanted at the native mitral valve 16 and the docking device delivery apparatus 50 (including the delivery shaft 54) has been removed from the patient 10 such that only the guidewire 40 and the guide catheter 30 remain inside the patient 10. In some examples, after removing the docking device delivery apparatus, the guidewire 40 can be advanced out of the guide catheter 30, through the implanted docking device 52 at the native mitral valve 16, and into the left ventricle 26 (FIG. 2A). As such, the guidewire 40 can help to guide the prosthetic valve delivery apparatus 60 through the annulus of the native mitral valve 16 and at least partially into the left ventricle 26.

As illustrated in FIG. 2B, the docking device 52 can comprise a plurality of helical turns that wrap around the leaflets 24 of the native mitral valve 16 (within the left ventricle 26). The implanted docking device 52 can have a more cylindrical shape than the annulus of the native mitral valve 16, thereby providing a geometry that more closely matches the shape or profile of the prosthetic heart valve to be implanted. As a result, the docking device 52 can provide a tighter fit, and thus a better seal, between the prosthetic heart valve and the native mitral valve 16, as described further below.

FIG. 3A depicts a fourth stage in the mitral valve replacement procedure where the user is delivering and/or implanting a prosthetic heart valve 62 within the docking device 52 using a prosthetic valve delivery apparatus 60.

As shown in FIG. 3A, the prosthetic valve delivery apparatus 60 can comprise a delivery shaft 64 and a handle 66. The delivery shaft 64 can extend distally from the handle 66. The delivery shaft 64 can be configured to extend into the patient’s vasculature 12 to deliver, implant, expand, and/or otherwise deploy the prosthetic heart valve 62 within the docking device 52 at the native mitral valve 16. The handle 66 can be configured to be gripped and/or otherwise held by the user to advance the delivery shaft 64 through the patient’s vasculature 12.

In some examples, the handle 66 can comprise one or more articulation members 68 that are configured to aid in navigating the delivery shaft 64 through the vasculature 12 and the heart 14. Specifically, the articulation members 68 can comprise one or more of knobs, buttons, wheels, and/or other types of physically adjustable control members that are configured to be adjusted by the user to flex, bend, twist, turn, and/or otherwise articulate a distal end portion of the delivery shaft 64 to aid in navigating the delivery shaft 64 through the vasculature 12 and into the left atrium 18 and left ventricle 26 of the heart 14.

In some examples, the prosthetic valve delivery apparatus 60 can include an expansion mechanism 65 that is configured to radially expand and deploy the prosthetic heart valve 62 at the implantation site. In some instances, as shown in FIG. 3A, the expansion mechanism 65 can comprise an inflatable balloon that is configured to be inflated to radially expand the prosthetic heart valve 62 within the docking device 52. The inflatable balloon can be coupled to the distal end portion of the delivery shaft 64.

In other examples, the prosthetic heart valve 62 can be self-expanding and can be configured to radially expand on its own upon removable of a sheath or capsule covering the radially compressed prosthetic heart valve 62 on the distal end portion of the delivery shaft 64. In still other examples, the prosthetic heart valve 62 can be mechanically expandable and the prosthetic valve delivery apparatus 60 can include one or more mechanical actuators (for example, the expansion mechanism) configured to radially expand the prosthetic heart valve 62.

As shown in FIG. 3A, the prosthetic heart valve 62 can be mounted around the expansion mechanism 65 (for example, the inflatable balloon) on the distal end portion of the delivery shaft 64, in a radially compressed configuration.

To navigate the distal end portion of the delivery shaft 64 to the implantation site, the user can insert the prosthetic valve delivery apparatus 60 (for example, the delivery shaft 64) into the patient 10 through the guide catheter 30 and over the guidewire 40. The user can continue to advance the prosthetic valve delivery apparatus 60 along the guidewire 40 (for example, through the vasculature 12) until the distal end portion of the delivery shaft 64 reaches the native mitral valve 16, as illustrated in FIG. 3A. More specifically, the user can advance the delivery shaft 64 of the prosthetic valve delivery apparatus 60 by gripping and exerting a force on (for example, pushing) the handle 66. While advancing the delivery shaft 64 through the vasculature 12 and the heart 14, the user can adjust the one or more articulation members 68 of the handle 66 to navigate the various turns, corners, constrictions, and/or other obstacles in the vasculature 12 and heart 14.

The user can advance the delivery shaft 64 along the guidewire 40 until the radially compressed prosthetic heart valve 62 mounted around the distal end portion of the delivery shaft 64 is positioned within the docking device 52 and the native mitral valve 16. In some examples, as shown in FIG. 3A, a distal end of the delivery shaft 64 and a least a portion of the radially compressed prosthetic heart valve 62 can be positioned within the left ventricle 26.

Once the radially compressed prosthetic heart valve 62 is appropriately positioned within the docking device 52 (FIG. 3A), the user can manipulate one or more actuation mechanisms of the handle 66 of the prosthetic valve delivery apparatus 60 to actuate the expansion mechanism 65 (for example, inflate the inflatable balloon), thereby radially expanding the prosthetic heart valve 62 within the docking device 52. In some examples, the user can lock the prosthetic heart valve 62 in its fully expanded position (for example, with a locking mechanism) to prevent the prosthetic heart valve 62 from collapsing.

FIG. 3B shows a fifth stage in the mitral valve replacement procedure where the prosthetic heart valve 62 in its radially expanded configuration and implanted within the docking device 52 in the native mitral valve 16. As shown in FIG. 3B, the prosthetic heart valve 62 can be received and retained within the docking device 52.

As also shown in FIG. 3B, after the prosthetic heart valve 62 has been fully deployed and implanted within the docking device 52 at the native mitral valve 16, the prosthetic valve delivery apparatus 60 (including the delivery shaft 64) can be removed from the patient 10 such that only the guidewire 40 and the guide catheter 30 remain inside the patient 10.

FIG. 4 depicts a sixth stage in the mitral valve replacement procedure, where the guidewire 40 and the guide catheter 30 have been removed from the patient 10. The docking device 52 can be configured to provide a seal between the prosthetic heart valve 62 and the leaflets 24 of the native mitral valve 16 to reduce paravalvular leakage around the prosthetic heart valve 62. Specifically, the docking device 52 can initially constrict the leaflets 24 of the native mitral valve 16. The prosthetic heart valve 62 can then push the leaflets 24 against the docking device 52 as it radially expands within the docking device 52. Thus, the docking device 52 and the prosthetic heart valve 62 can be configured to sandwich the leaflets 24 of the native mitral valve 16 when the prosthetic heart valve 62 is expanded within the docking device 52. In this way, the docking device 52 can provide a seal between the leaflets 24 of the native mitral valve 16 and the prosthetic heart valve 62 to reduce paravalvular leakage around the prosthetic heart valve 62.

In some examples, one or more of the docking device delivery apparatus 50, the prosthetic valve delivery apparatus 60, and/or the guide catheter 30 can comprise one or more fluid ports that are configured to supply flushing fluid to the lumens thereof to prevent and/or reduce the likelihood of blood clot (for example, thrombus) formation. Example fluid ports that can be used to inject flushing fluid into a docking device delivery apparatus are described further below.

Although FIGS. 1-4 specifically depict a mitral valve replacement procedure, it should be appreciated that the same and/or similar procedure may be utilized to replace other heart valves (for example, tricuspid, pulmonary, and/or aortic valves). Further, the same and/or similar delivery apparatuses (for example, docking device delivery apparatus 50, prosthetic valve delivery apparatus 60, guide catheter 30, and/or guidewire 40), docking devices (for example, docking device 52), replacement heart valves (for example, prosthetic heart valve 62), and/or components thereof may be utilized for replacing these other heart valves.

For example, when replacing a native tricuspid valve, the user may also access the right atrium 20 via a femoral vein but may not need to cross the atrial septum 22 into the left atrium 18. Instead, the user may leave the guidewire 40 in the right atrium 20 and perform the same and/or similar docking device implantation process at the tricuspid valve. Specifically, the user may push the docking device 52 out of the delivery shaft 54 around the ventricular side of the tricuspid valve leaflets, release the remaining portion of the docking device 52 from the delivery shaft 54 within the right atrium 20, and then remove the delivery shaft 54 of the docking device delivery apparatus 50 from the patient 10. The user may then advance the guidewire 40 through the tricuspid valve into the right ventricle and perform the same and/or similar prosthetic heart valve implantation process at the tricuspid valve, within the docking device 52. Specifically, the user may advance the delivery shaft 64 of the prosthetic valve delivery apparatus 60 through the patient’s vasculature along the guidewire 40 until the prosthetic heart valve 62 is positioned or disposed within the docking device 52 and the tricuspid valve. The user may then expand the prosthetic heart valve 62 within the docking device 52 before removing the prosthetic valve delivery apparatus 60 from the patient 10. In another example, the user may perform the same and/or similar process to replace the aortic valve but may access the aortic valve from the outflow side of the aortic valve via a femoral artery.

Further, although FIGS. 1-4 depict a mitral valve replacement procedure that accesses the native mitral valve 16 from the left atrium 18 via the right atrium 20 and femoral vein, it should be appreciated that the native mitral valve 16 may alternatively be accessed from the left ventricle 26. For example, the user may access the native mitral valve 16 from the left ventricle 26 via the aortic valve by advancing one or more delivery apparatuses through an artery to the aortic valve, and then through the aortic valve into the left ventricle 26.

Additional examples of the docking device delivery apparatus, including its variants, and methods of implanting a docking device and implanting a prosthetic valve within the docking device are described in PCT Patent Application Publication Nos. WO 2020/247907 and WO 2022/087336, and U.S. Patent Publication Nos. US2018/0318079, US2018/0263764, and US2018/0177594, which are all incorporated by reference herein in their entireties.

### Overview of Docking Devices

Docking devices according to examples of the disclosure can, for example, provide a stable anchoring site, landing zone, or implantation zone at the implant site in which prosthetic valves can be expanded or otherwise implanted. Many of the disclosed docking devices comprise a circular or cylindrically-shaped portion, which can (for example) allow a prosthetic heart valve comprising a circular or cylindrically-shaped valve frame to be expanded or otherwise implanted into native locations with naturally circular cross-sectional profiles and/or in native locations with naturally with non-circular cross sections. In addition to providing an anchoring site for the prosthetic valve, the docking devices can be sized and shaped to cinch or draw the native valve (for example, mitral, tricuspid, etc.) anatomy radially inwards. In this manner, one of the main causes of valve regurgitation (for example, functional mitral regurgitation), specifically enlargement of the heart (for example, enlargement of the left ventricle, etc.) and/or valve annulus, and consequent stretching out of the native valve (for example, mitral, etc.) annulus, can be at least partially offset or counteracted. Some examples of the docking devices further include features which, for example, are shaped and/or modified to better hold a position or shape of the docking device during and/or after expansion of a prosthetic valve therein. By providing such docking devices, replacement valves can be more securely implanted and held at various valve annuluses, including at the mitral valve annulus which does not have a naturally circular cross-section.

In some instances, a docking device can comprise a paravalvular leakage (PVL) guard (also referred to herein as “a guard member”). The PVL guard can, for example, help reduce regurgitation and/or promote tissue ingrowth between the native tissue and the docking device.

The PVL guard can, in some examples, be movable between a delivery configuration and a deployed configuration. When the PVL guard is in the delivery configuration, the PVL guard can extend along and adjacent the coil. When the PVL guard is in the deployed configuration, the PVL guard can form a helical shape rotating about a central longitudinal axis of the coil and at least a segment of the PVL guard can extend radially away from the coil.

In certain examples, the PVL guard can cover or surround a portion of a coil of the docking device. As described more fully below, such PVL guard can move from a radially compressed (and axially elongated) state to a radially expanded (and axially foreshortened) state.

In certain examples, a distal end of the PVL guard can be fixedly attached to the coil while a proximal end of the PVL guard can be axially movable relative to the coil.

### Exemplary Docking Devices

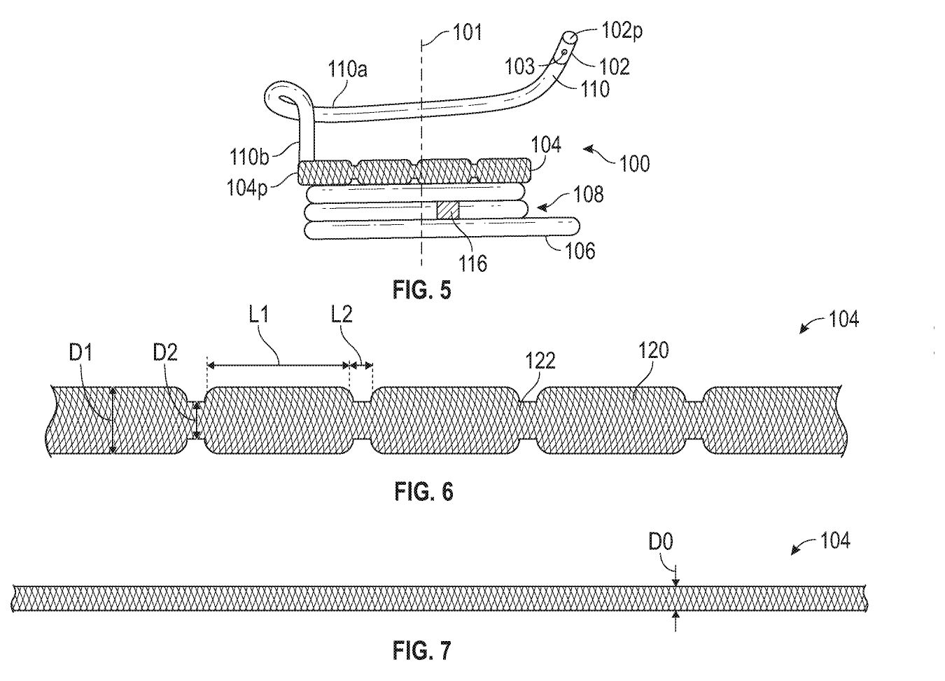

FIG. 5 shows a docking device 100, according to one example. The docking device 100 can, for example, be implanted within a native valve annulus. The docking device 100 can be configured to receive and secure a prosthetic valve (for example, prosthetic heart valve 62), thereby securing the prosthetic valve at the native valve annulus.

The docking device 100 can comprise a coil 102 and a PVL guard or guard member 104 covering at least a portion of the coil 102. In certain examples, the coil 102 can include a shape memory material (for example, nickel titanium alloy or “Nitinol”) such that the docking device 100 (and the coil 102) can move from a substantially straight configuration (also referred to as “delivery configuration”) when disposed within a delivery sheath of a delivery apparatus (for example, docking device delivery apparatus 50) to a helical configuration (also referred to as “deployed configuration,” as shown in FIG. 5) after being removed from the delivery sheath.

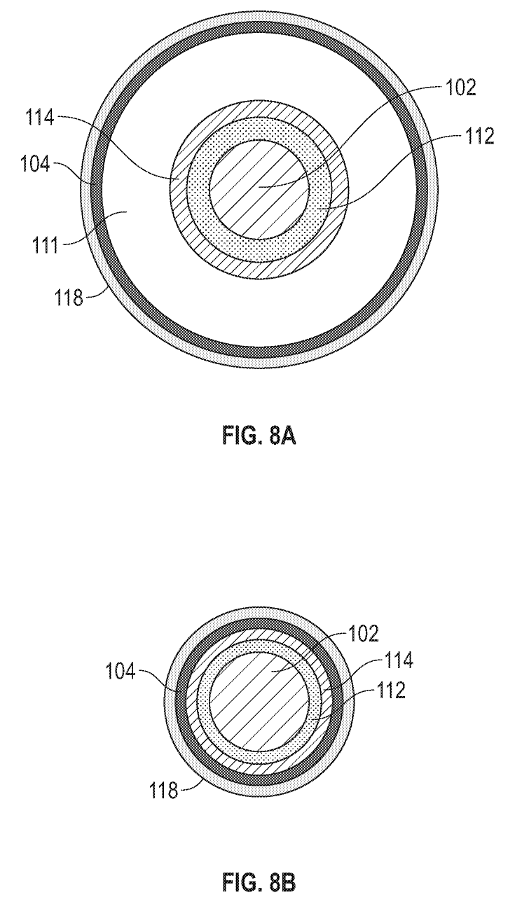

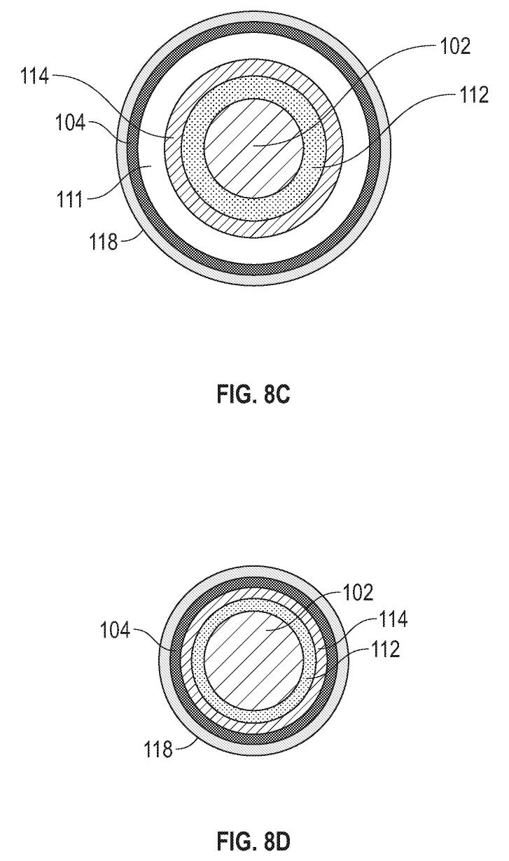

During delivery of the docking device and after initial deployment of the docking device at the implantation site, the guard member 104 can be retained in a radially compressed state by a dock sleeve of the delivery apparatus such that no void space (or gap, or cavity) is formed between the guard member 104 and the coil 102 (see, for example, FIGS. 8B and 8D). After the docking device 100 is deployed at the implantation site, the dock sleeve can be removed so as to expose the guard member 104, thereby allowing the guard member 104 to move to a radially expanded state such that a void space or gap is formed between the guard member and the coil (see, for example, FIGS. 8A and 8C). FIG. 6 shows a portion of the guard member 104 in the radially expanded state, and FIG. 7 shows a portion of the guard member 104 in the radially compressed state.

In certain examples, when the docking device 100 is in the deployed configuration and the guard member 104 is in the radially expanded state, the guard member 104 can extend circumferentially relative to a central longitudinal axis 101 of the docking device 100 from 180 degrees to 400 degrees, or from 210 degrees to 330 degrees, or from 250 degrees to 290 degrees, or from 260 degrees to 280 degrees (for example, 270 degrees) relative to the central longitudinal axis 101. In other words, the guard member 104 can extend circumferentially from about one half of a revolution (for example, 180 degrees) around the central longitudinal axis 101 in some examples to more than a full revolution (for example, 400 degrees) around the central longitudinal axis 101 in other examples, including various ranges in between. As used herein, a range (for example, from 180 degrees to 400 degrees, and between 180 degrees and 400 degrees) includes the endpoints of the range (for example, 180 degrees and 400 degrees).

The coil 102 has a proximal end 102p and a distal end, which also respectively define the proximal and distal ends of the docking device 100. When being disposed within the delivery sheath (for example, during delivery of the docking device into the vasculature of a patient), a body of the coil 102 between the proximal end 102p and distal end can form the generally straight delivery configuration (that is, without any coiled or looped portions, but can be flexed or bent) so as to maintain a small radial profile when moving through a patient’s vasculature. After being removed from the delivery sheath and deployed at an implant position, the coil 102 can move from the delivery configuration to the helical deployed configuration and wrap around native tissue adjacent the implant position. For example, when implanting the docking device at the location of a native valve, the coil 102 can be configured to surround native leaflets of the native valve (and the chordae tendineae that connects native leaflets to adjacent papillary muscles, if present).

The docking device 100 can be releasably coupled to a delivery apparatus (for example, docking device delivery apparatus 50). For example, in certain examples, the docking device 100 can be coupled to the delivery apparatus via a release suture that can be configured to be tied to the docking device 100 and cut for removal. In one example, the release suture can be tied to the docking device 100 through an eyelet or eyehole 103 located adjacent the proximal end 102p of the coil. In another example, the release suture can be tied around a circumferential recess that is located adjacent the proximal end 102p of the coil 102.

In some examples, the docking device 100 in the deployed configuration can be configured to fit at the mitral valve position. In other examples, the docking device 100 can also be shaped and/or adapted for implantation at other native valve positions as well, such as at the tricuspid valve. As described herein, the geometry of the docking device 100 can be configured to engage the native anatomy, which can, for example, provide for increased stability and reduction of relative motion between the docking device 100, the prosthetic valve docked therein, and/or the native anatomy. Reduction of such relative motion can, among other things, prevent material degradation of components of the docking device 100 and/or the prosthetic valve docked therein and/or prevent damage or trauma to the native tissue.

As shown in FIG. 5, the coil 102 in the deployed configuration can include a leading turn 106 (or “leading coil”), a central region 108, and a stabilization turn 110 (or “stabilization coil”) around the central longitudinal axis 101. The central region 108 can possess one or more helical turns having substantially equal inner diameters. The leading turn 106 can extend from a distal end of the central region 108 and has a diameter greater than the diameter of the central region 108 (in one or more configurations). The stabilization turn 110 can extend from a proximal end of the central region 108 and has a diameter greater than the diameter of the central region 108 (in one or more configurations).

In certain examples, the central region 108 can include a plurality of helical turns (for example, the docking device depicted in FIG. 5 has three helical turns in the central region 108). Some of the helical turns in the central region 108 can be full turns (that is, rotating 360 degrees). In some examples, the most proximal turn and/or the most distal turn can be partial turns (for example, rotating less than 360 degrees, such as 180 degrees, 270 degrees, etc.).

The size of the docking device 100 can be generally selected based on the size of the desired prosthetic valve to be implanted into the patient. In certain examples, the central region 108 can be configured to retain a radially expandable prosthetic valve. For example, the inner diameter of the helical turns in the central region 108 can be configured to be smaller than an outer diameter of the prosthetic valve when the prosthetic valve is radially expanded so that additional radial force can act between the central region 108 and the prosthetic valve to hold the prosthetic valve in place. The helical turns in the central region 108 can also be referred to herein as “functional turns.”

The stabilization turn 110 can be configured to help stabilize the docking device 100 in the desired position. For example, the radial dimension of the stabilization turn 110 can be significantly larger than the radial dimension of the coil in the central region 108, so that the stabilization turn 110 can flare or extend sufficiently outwardly so as to abut or push against the walls of the circulatory system, thereby improving the ability of the docking device 100 to stay in its desired position prior to the implantation of the prosthetic valve. In some examples, the diameter of stabilization turn 110 is desirably larger than the native annulus, native valve plane, and/or native chamber for better stabilization. In some examples, the stabilization turn 110 can be a full turn (that is, rotating about 360 degrees). In some examples, the stabilization turn 110 can be a partial turn (for example, rotating between about 180 degrees and about 270 degrees).

In one particularly example, when implanting the docking device 100 at the native mitral valve location, the functional turns in the central region 108 can be disposed substantially in the left ventricle and the stabilization turn 110 can be disposed substantially in the left atrium. The stabilization turn 110 can be configured to provide one or more points or regions of contact between the docking device 100 and the left atrial wall, such as at least three points of contact in the left atrium or complete contact on the left atrial wall. In certain examples, the points of contact between the docking device 100 and the left atrial wall can form a plane that is approximately parallel to a plane of the native mitral valve.

In some examples, the stabilization turn 110 can have an atrial portion (covered by the guard member 104 in FIG. 5) in connection with the central region 108, a stabilization portion 110a adjacent to the proximal end 102p of the coil 102, and an ascending portion 110b located between the atrial portion and the stabilization portion 110a. Both the atrial portion and the stabilization portion 110a can be generally parallel to the helical turns in the central region 108, whereas the ascending portion 110b can be oriented to be angular relative to the atrial portion and the stabilization portion 110a. For example, in certain examples, the ascending portion 110b and the stabilization portion 110a can form an angle from about 45 degrees to about 90 degrees (inclusive). When implanting the docking device 100 at the native mitral valve location, the atrial portion can be configured to abut the posterior wall of the left atrium and the stabilization portion 110a can be configured to flare out and press against the anterior wall of the left atrium.

As noted above, the leading turn 106 can have a larger radial dimension than the helical turns in the central region 108. The leading turn 106 can help more easily guide the coil 102 around and/or through the chordae tendineae and/or adequately around all native leaflets of the native valve (for example, the native mitral valve, tricuspid valve, etc.). For example, once the leading turn 106 is navigated around the desired native anatomy, the remaining coil (such as the functional turns) of the docking device 100 can also be guided around the same features. In some examples, the leading turn 106 can be a full turn (that is, rotating about 360 degrees). In some examples, the leading turn 106 can be a partial turn (for example, rotating between about 180 degrees and about 270 degrees). When a prosthetic valve is radially expanded within the central region 108 of the coil, the functional turns in the central region 108 can be further radially expanded. As a result, the leading turn 106 can be pulled in the proximal direction and become a part of the functional turn in the central region 108.

As shown in FIGS. 8A-8D, at least a portion of the coil 102 can be surrounded by an inner cover 112. The inner cover 112 can have a tubular shape and thus can also be referred to as a “tubular member.” In certain examples, the inner cover 112 can cover an entire length of the coil 102. In certain examples, the inner cover 112 covers only selected portion(s) of the coil 102.

In certain examples, the inner cover 112 can be coated on and/or bonded on the coil 102. In certain examples, the inner cover 112 can be a cushioned, padded-type layer protecting the coil 102. The inner cover 112 can be constructed of various native and/or synthetic materials. In one particularly example, the inner cover 112 can include expanded polytetrafluoroethylene (ePTFE). In certain examples, the inner cover 112 is configured to be fixedly attached to the coil 102 (for example, by means of textured surface resistance, suture, glue, thermal bonding, or any other means) so that relative axial movement between the inner cover 112 and the coil 102 is restricted or prohibited.

In some examples, the docking device 100 can also include a retention member 114 (see, for example, FIGS. 8A-8D) surrounding at least a portion of the coil 102 (and the inner cover 112) and at least being partially covered by the guard member 104. In some examples, the inner cover 112 can extend through an entire length of the retention member 114. In some examples, at least a portion of the inner cover 112 may not be surrounded by the retention member 114.

In some examples, the retention member 114 can comprise a braided material. In some examples, the retention member 114 can include a woven material. In addition, the retention member 114 can provide a surface area that encourages or promotes tissue ingrowth and/or adherence, and/or reduce trauma to native tissue. For example, in certain instances, the retention member 114 can have a textured outer surface configured to promote tissue ingrowth. In certain instances, the retention member 114 can be impregnated with growth factors to stimulate or promote tissue ingrowth.

In some examples, at least a proximal end portion of the retention member 114 can extend out of (that is, positioned proximal to) a proximal end 104p of the guard member 104. In one particular example, the proximal end of the retention member 114 can be disposed at or adjacent the ascending portion 110b of the coil 102. In some examples, at least a distal end portion of the retention member 114 can extend out of (that is, positioned distal to) a distal end of the guard member 104. In one specific example, a distal end of the retention member 114 can be positioned adjacent the leading turn 106. In some examples, the retention member 114 can cover the functional turns of the coil 102 in the central region 108. Thus, when the docking device 100 is deployed at the native valve and the prosthetic valve is radially expanded within the docking device 100, the retention member 114 at the central region 108 can frictionally engage the prosthetic valve. In some examples, the retention member 114 can be completely covered by the guard member 104.

In some examples, the retention member 114 can be configured to interact with the guard member 104 to limit or resist axial movement of the guard member 104 relative to the coil 102. For example, the proximal end 104p of the guard member 104 can have an inner diameter that is about the same as an outer diameter of the retention member 114. As such, an inner surface of the guard member 104 at the proximal end 104p can frictionally engage with the retention member 114 so that axial movement of the proximal end 104p of the guard member 104 relative to the coil 102 can be impeded by a frictional force exerted by the retention member 114.

In some examples, when the docking device 100 is in the deployed configuration, the guard member 104 can be configured to cover a portion (for example, the atrial portion) of the stabilization turn 110 of the coil 102. In certain examples, the guard member 104 can be configured to cover at least a portion of the central region 108 of the coil 102 (for example, a portion of the most proximal turn). In certain examples, the guard member 104 can extend over a majority (or even an entirety) of the functional turns in the central region 108. In one example, when the docking device 100 is deployed at a native atrioventricular valve, the guard member 104 does not extend into the ascending portion 110b to improve the durability of the guard member (for example, to reduce the likelihood of kinking of the guard member).

In certain circumstances, the retention member 114, by frictional engagement with the proximal end 104p of the guard member, can prevent the guard member 104 from extending into the ascending portion 110b of the coil 102.

As described herein, the guard member 104 can radially expand so as to help preventing and/or reducing paravalvular leakage. Specifically, the guard member 104 can be configured to radially expand such that an improved seal is formed closer to and/or against a prosthetic valve deployed within the docking device 100. In some examples, the guard member 104 can be configured to prevent and/or inhibit leakage at the location where the docking device 100 crosses between leaflets of the native valve (for example, at the commissures of the native leaflets). For example, without the guard member 104, the docking device 100 may push the native leaflets apart at the point of crossing the native leaflets and allow for leakage at that point (for example, along the docking device or to its sides). However, the guard member 104 can be configured to expand to cover and/or fill any opening at that point and inhibit leakage along the docking device 100. Additional features of the guard member 104 helping to reduce paravalvular leakage are described further below.

In various examples, the guard member 104 can help covering an atrial side of an atrioventricular valve to prevent and/or inhibit blood from leaking through the native leaflets, commissures, and/or around an outside of the prosthetic valve by blocking blood in the atrium from flowing in an atrial to ventricular direction (that is, antegrade blood flow)—other than through the prosthetic valve. Positioning the guard member 104 on the atrial side of the valve can additionally or alternatively help reduce blood in the ventricle from flowing in a ventricular to atrial direction (that is, retrograde blood flow).

In some examples, the guard member 104 can be positioned on a ventricular side of an atrioventricular valve to prevent and/or inhibit blood from leaking through the native leaflets, commissures, and/or around an outside of the prosthetic valve by blocking blood in the ventricle from flowing in a ventricular to atrial direction (that is, retrograde blood flow). Positioning the guard member 104 on the ventricular side of the valve can additionally or alternatively help reduce blood in the atrium from flowing in the atrial direction to ventricular direction (that is, antegrade blood flow)—other than through the prosthetic valve.

In certain examples, the docking device 100 can include at least one radiopaque marker configured to provide visual indication about the location of the docking device 100 relative to its surrounding anatomy, and/or the amount of radial expansion of the docking device 100 (for example, when a prosthetic valve is subsequently deployed in the docking device 100) under fluoroscopy. For example, one or more radiopaque markers can be placed on the coil 102. In one particularly example, a radiopaque marker 116 can be disposed at the central region 108 of the coil. In some examples, one or more radiopaque markers can be placed on the inner cover 112, the guard member 104, and/or other components of the docking device. In some examples, the docking device 100 can also have one or more radiopaque markers located distal to the ascending portion 110b of the coil 102 (for example, to ensure the proximal end 104p of the guard member 104 does not extend into the ascending portion 110b).

Further details of the guard member 104 are described in the section “Exemplary PVL Guard” below.

Additional examples of the docking device and its variants, including various examples of the coil, the guard member, the inner cover, the retention member, and other components of the docking device, are described in PCT Patent Application Publication No. WO/2020/247907, the entirety of which is incorporated by reference herein. Example methods of assembling the guard member are described in PCT Patent Application Publication No. WO 2022/087336 and International Patent Application No. PCT/US2022/045376, each of which is incorporated by reference herein in its entirety.

### Exemplary PVL Guards

In certain examples, the guard member 104 can include a braided structure, such as a braided wire mesh or lattice. In certain examples, the guard member 104 can include a shape memory material that is shape set and/or pre-configured to expand to a particular shape and/or size when unconstrained (for example, when deployed at a native valve location). For example, the guard member 104 can have a braided structure containing a shape memory alloy with super-elastic properties, such as Nitinol. In certain examples, the guard member 104 can have a braided structure containing a ternary shape memory alloy with Superelastic properties, such as NiTiX where X can be chromium (Cr), cobalt (Co), zirconium (Zr), hafnium (Hf), etc.

In certain examples, the guard member 104 can comprise a metallic material that does not have the shape memory properties. Examples of such metallic material include cobalt-chromium, stainless steel, etc. In one specific example, the guard member 104 can comprise nickel-free austenitic stainless steel in which nickel can be completely replaced by nitrogen. In another specific example, the guard member 104 can comprise cobalt-chromium or cobalt-nickel-chromium-molybdenum alloy with significantly low density of titanium.

The number of wires (or fibers, strands, or the like) forming the braided structure can be selected to achieve a desired elasticity and/or strength of the guard member 104. In certain examples, the number of wires used to braid the guard member 104 can range from 16 to 128 (for example, 32 wires, 48 wires, 64 wires, 96 wires, etc.). In certain examples, the braid density can range from 20 picks per inch (PPI) to 70 PPI, or from 25 PPI to 65 PPI. In one specific example, the braid density is about 36 PPI. In another specific example, the braid density is about 40 PPI. In certain examples, the diameter of the wires can range from about 0.002 inch to about 0.004 inch. In one particularly example, the diameter of the wires can be about 0.003 inch.

In another example, the guard member 104 can be a combination of braided wires (which can include a shape memory material or non-shape memory material) and a polymeric material and/or textile (for example, polyethylene terephthalate (PET), polytetrafluoroethylene (PTFE), polyether ether ketone (PEEK), thermoplastic polyurethane (TPU), etc.). For example, the guard member 104 can include a braided wire frame embedded in a polymeric material.

In some examples, the guard member 104 can include a braided metallic wire frame coated with an elastomer (for example, ePTFE, TPU, or the like), which can elastically deform as the braided wire frame expands and/or compresses. In some examples, the guard member 104 can comprise a braid and/or weave that includes one or more metallic wires and one or more polymeric fibers. In other words, the metallic wires and the polymeric fibers can be interwoven together to define a braided structure. In some instances, the polymeric fibers can have the same or about the same diameter as the metallic wires. In other instances, the polymeric fibers can have a smaller diameter (for example, microfibers) than the metallic wires, or vice versa.

The guard member 104 extends radially outwardly from the coil 102 and is movable between a radially compressed (and axially elongated) state and a radially expanded (and axially foreshortened) state. In other words, the guard member 104 can axially foreshorten when it moves from the radially compressed state to the radially expanded state and can axially elongate when it moves from the radially expanded state to the radially compressed state.

In some examples, a distal end of the guard member 104 can be fixedly coupled to the coil 102 (for example, via suturing, gluing, or the like), and the proximal end 104p of the guard member 104 can be axially movable relative to the coil 102. Thus, the proximal end 104p of the guard member 104 can slide axially over the inner cover 112 in a distal direction when guard member 104 moves from the radially compressed state to the radially expanded state. In other words, the proximal end 104p of the guard member 104 can be disposed closer to the proximal end 102p of the coil 102 when the guard member 104 is in the radially compressed state than in the radially expanded state.

Alternatively, the proximal end 104p of the guard member 104 can be fixedly coupled to the coil 102, whereas the distal end of the guard member 104 can be axially movable relative to the coil 102.

In certain examples, as shown in FIGS. 6-7, the guard member 104 includes a plurality of body portions 120 and one or more neck portions 122 connecting the plurality of body portions 120.

As depicted in FIG. 6, when the guard member 104 is in the radially expanded state, a diameter (D2) of the neck portions 122 is smaller than a diameter (D1) of the body portions 120. As depicted in FIG. 7, when the guard member 104 is in the radially compressed state, the neck portions 122 and the body portions 120 can have a same compressed diameter (D0). Generally, D1 is greater than D2, and D2 is greater than D0.

In certain examples, D0 is between 1 mm and 4 mm, or between 2 mm and 3 mm, all inclusive.

In certain examples, D2 is between 5 mm and 6.5 mm, inclusive.

In certain examples, D1 is between 7 mm and 10 mm, inclusive.

In certain examples, D2 and D1 can define a diameter ratio D2/D1, which is between 0.5 and 0.95, or between 0.75 and 0.9, all inclusive.

In certain examples, D1 and D0 can define a first compression ratio D1/D0, and D2 and D0 can define a second compression ratio D2/D0. Because D1 is larger than D2, the first compression ratio D1/D0 is larger than the second compression ratio D2/D0.

In certain examples, the first compression ratio D1/D0 is between 2 and 6, inclusive.

In certain examples, the second compression ratio D2/D0 is between 1.5 and 4, inclusive.

When the guard member 104 is in the radially expanded state, an axial length (L2) of the neck portions 122 can be smaller than an axial length (L1) of the body portions 120.

In certain examples, L2 is between 1.5 mm and 2.5 mm, inclusive.

In certain examples, L1 is between 8 mm and 10 mm, inclusive.

In certain examples, L2 and L1 can define a length ratio L2/L1, which is between 0.1 and 0.3, or between 0.2 and 0.25, all inclusive.

In certain examples, the number of body portions 120 is between two and ten, or between four and six, all inclusive. In certain examples, the number of neck portions 122 can be one less than the number of body portions.

In the depicted examples, different body portions 120 have the same or substantially similar size. In other examples, the body portions 120 can have varying sizes (for example, different body portions can have different axial lengths and/or different diameters). Similarly, different neck portions 122 can have the same size or difference sizes.

In some examples, as depicted in FIGS. 8A-8D, the docking device 100 can include an outer cover 118 surrounding an outer surface of the guard member 104. In some circumstances, the outer cover 118 can be deemed as a part of the guard member 104 covering an outer surface of the braided wire frame.

As described herein, the outer cover 118 can be configured to be so elastic that when the guard member 104 moves from the radially compressed (and axially elongated) state to the radially expanded (and axially foreshortened) state, the outer cover 118 can also radially expand and axially foreshorten together with the guard member 104.

In certain examples, the outer cover 118 can be configured to be atraumatic to native tissue and/or promote tissue ingrowth into the outer cover 118. For example, the outer cover 118 can have pores to encourage tissue ingrowth. In another example, the outer cover 118 can be impregnated with growth factors to stimulate or promote tissue ingrowth, such as transforming growth factor alpha (TGF-alpha), transforming growth factor beta (TGF-beta), basic fibroblast growth factor (bFGF), vascular epithelial growth factor (VEGF), and combinations thereof. The outer cover 118 can be constructed of any suitable material, including foam, cloth, fabric, and/or polymer, which is flexible to allow for compression and expansion of the outer cover 118. In one example, the outer cover 118 can include a fabric layer constructed from a thermoplastic polymer material, such as polyethylene terephthalate (PET).

In certain examples, the outer cover 118 can be configured to engage with the prosthetic valve deployed within the docking device 100 so as to form a seal and reduce paravalvular leakage between the prosthetic valve and the docking device 100 after the guard member 104 is radially expanded. The outer cover 118 can also be configured to engage with the native tissue (for example, the native annulus and/or native leaflets) to reduce PVL between the docking device and/or the prosthetic valve and the native tissue.

When the guard member 104 is in the radially expanded state, the wire frame of the guard member 104 expands radially outwardly so as to create a void space (or gap, or cavity) 111 between the guard member 104 and the coil 102. In the example depicted in FIGS. 8A and 8C, the void space 111 exists between the guard member 104 and the retention member 114. Because D2 is smaller than D1, the cross-sectional area of the void space 111 in the neck portions 122 is smaller than that in the body portions 120.

In some examples, when the guard member 104 is in the radially compressed state, no void space (or gap, or cavity) exists between the guard member 104 and the coil 102. In the example depicted in FIGS. 8B and 8D, only the retention member 114 and the inner cover 112 separate the coil 102 from the guard member 104.

In some examples, the guard member 104 in the radially expanded state can be generally coaxial with (or symmetric about) the coil 102, as depicted in FIGS. 8A-8D, when no prosthetic valve is radially expanded within the coil 102.

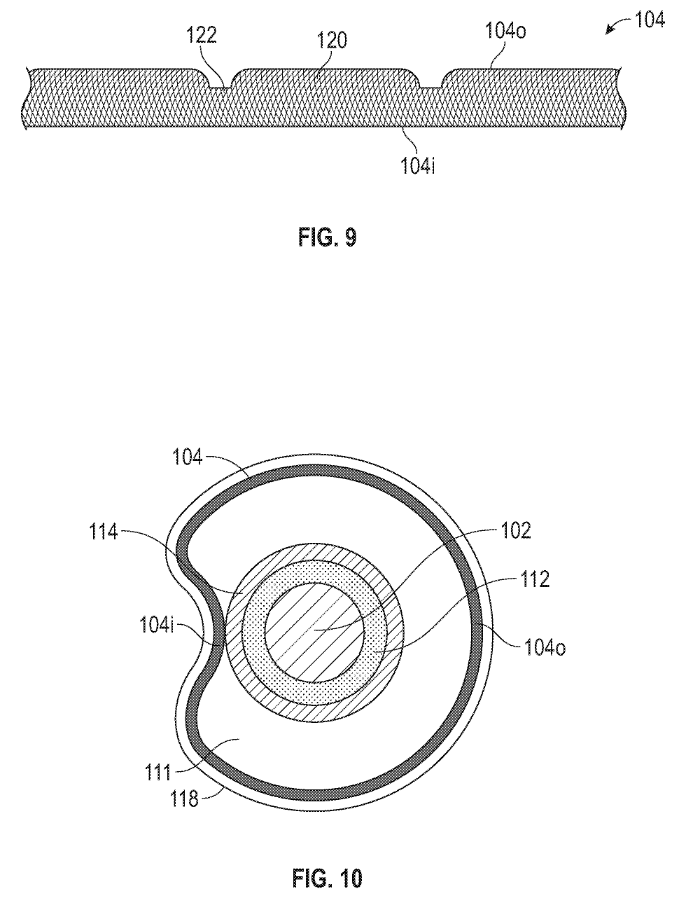

In some examples, when a prosthetic valve is radially expanded within the central region 108 of the docking device 100, the prosthetic valve can radially compress an inner side 104i of at least a portion of the guard member 104 so as to cause the guard member 104 to move from the radially expanded state to a semi-expanded state. As depicted in FIGS. 9-11, at the semi-expanded state, the inner side 104i of the portion of the guard member, compressed by the prosthetic valve, can move closer to the coil 102 whereas an outer side 104o of the portion of the guard member may retain its position relative to the coil 102. As a result, the portion of the guard member compressed by the prosthetic valve may not be coaxial with (or symmetric about) the coil 102.

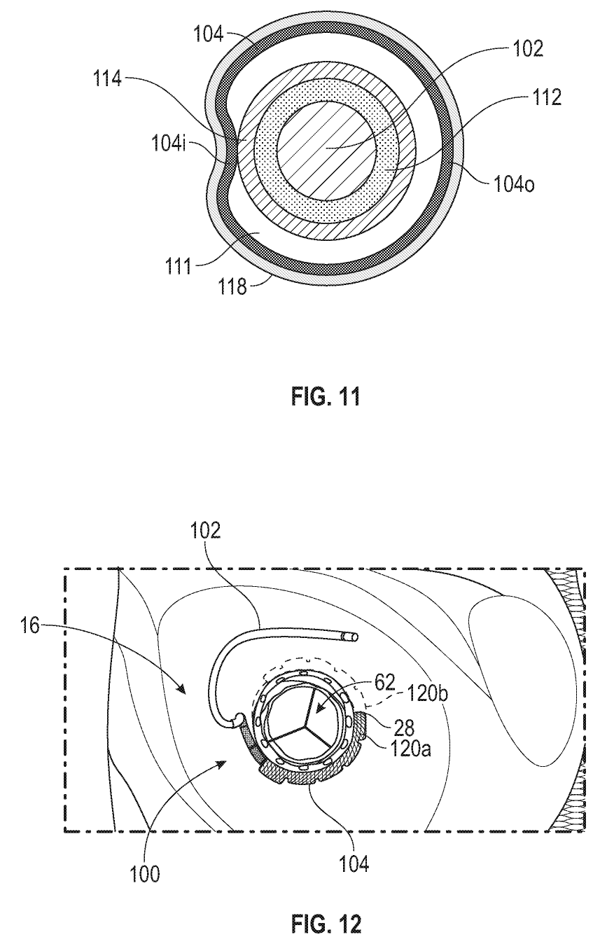

For example, FIG. 9 shows a portion of the guard member 104 that is radially compressed by a prosthetic valve (the inner side 104i is shown as the bottom edge of the guard member in FIG. 9). Cross-sectional views of the body portions 120 and neck portions 122 are respectively shown in FIGS. 10-11 (the inner side 104i is shown as the left side of the guard member in FIGS. 10-11). In the depicted example, the inner side 104i of the portion of the guard member is pressed radially inwardly (by a prosthetic valve) such that the retention member 114 (or the inner cover 112 if the retention member 114 is absent) can contact the inner side 104i and close the gap therebetween. In other examples, there can still be a gap between the inner side 104i and the retention member 114 (or the inner cover 112) when the guard member 104 is in the semi-expanded state, but the gap in the semi-expanded state is reduced compared to the gap when the guard member 104 is in the radially expanded state.

In the depicted example, the outer side 104o of the portion of the guard member 104 remains about the same position (relative to the coil 102) as it is in the radially expanded state (for example, the outer side 104o remains radially spaced away from the retention member 114 and the inner cover 112). In other examples, the outer side 104o of the guard member 104 may also be pressed radially inwardly by surrounding native tissues, thereby moving the outer side 104o closer to the coil 102.

In some examples, the axial length of the neck portions 122 can be configured to match the thickness of a commissure of a native valve. For example, each of the neck portions can be sized to receive a medial commissure of a native mitral valve. When deploying the docking device 100, the position and/or orientation of the docking device 100 can be adjusted so that at least one of the neck portions 122 can be aligned with a commissure of a native valve. As a result, the commissure of the native valve can be positioned between two adjacent body portions 120 of the guard member 104.

For example, FIG. 12 shows the docking device 100 deployed at the annulus of a native mitral valve 16 and a prosthetic valve 62 is radially expanded within the docking device 100 (thus, the guard member 104 can be in the semi-expanded state). In this example, the implantation site is a mitral annulus that separates the left atrium from the left ventricle (the view is from the left atrial side and the left ventricle is behind the mitral annular plane). A medial commissure 28 of the native mitral valve is received within one of the neck portions 122 of the guard member 104 and sandwiched between two adjacent body portions 120a, 120b of the guard member 104. The body portion 120a is located on the atrial side and the body portion 120b is located on the ventricle side. As such, the two body portions 120a, 120b can effectively function as plugs on both sides of the mitral valve 16, preventing paravalvular leakage through the medial commissure 28.

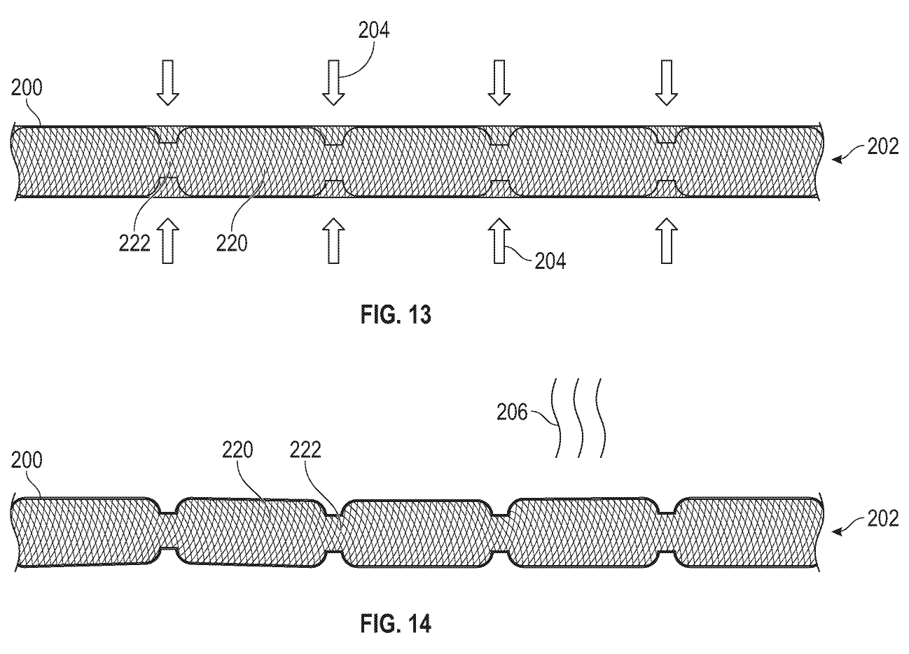

FIGS. 13-14 schematically illustrate a process of making the guard member 104.

As shown in FIG. 13, a layer of metal wires 200 containing a shape memory material (for example, Nitinol) can be braided over a mandrel 202 (or fixture) to form an initial state of the guard member 104. The guard member 104 in the initial state can have a generally cylindrical shape. The mandrel 202 can be configured to have a shape or contour that matches that of a desired final product of the guard member 104 in the radially expanded state. For example, FIG. 13 shows that the mandrel 202 includes a plurality of body portions 220 and one or more neck portions 222 connecting the plurality of body portions 220. The neck portions 222 has a smaller diameter than the body portions 220.

After the layer of metal wires 200 is braided over the mandrel 202, radial pressure (indicated by the arrows 204) can be applied, for example, by a clamping device, to portions of the metal wires 200 over the neck portions 222 of the mandrel 202 to transform the guard member 104 from the initial state to a temporarily deformed state. The guard member 104 in the temporarily deformed state generally conforms to a shape of the mandrel 202 (see, FIG. 14).

Then, as shown in FIG. 14, heat 206 can be applied to the guard member 104 at a predetermined temperature over a predetermined duration to transform the guard member 104 from the temporarily deformed state to a permanently deformed state. The controlled heating can shape set the layer of metal wires 200 so that the guard member 104 in the permanently deformed state retains the shape of the mandrel 202 when no external force is applied to the guard member 104. The layer of metal wires can then be cut to length and removed from the mandrel 202, thereby producing the guard member 104 of the desired shape.

### Exemplary Prosthetic Valves

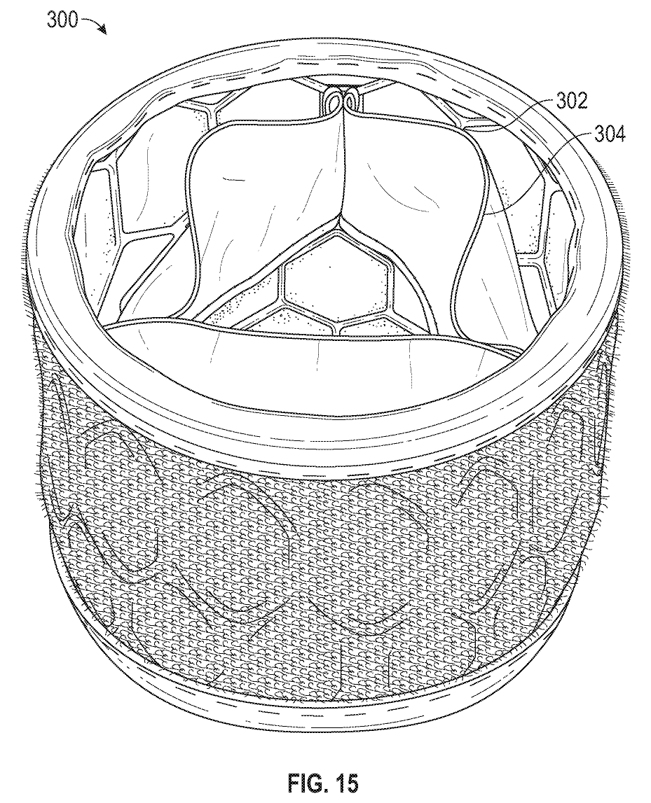

FIG. 15 shows a prosthetic heart valve 300, which can be one specific example of the prosthetic valve 62 described above. As shown, the heart valve 300 comprises a frame, or stent, 302 and a leaflet structure 304 supported by the frame. In some examples, the prosthetic heart valve 300 is adapted to be implanted in the native aortic valve and can be implanted in the body using, for example, the delivery apparatus 10 described above. The prosthetic valve 300 can also be implanted within the body using any of the other delivery apparatuses described herein.

In some examples, the frame 302 comprises a plastically expandable material, which can be metal alloys, polymers, or combinations thereof. Example metal alloys can comprise one or more of the following: nickel, cobalt, chromium, molybdenum, titanium, or other biocompatible metal. In some examples, the frame 302 can comprise stainless steel. In some examples, the frame 302 can comprise cobalt-chromium. In some examples, the frame 302 can comprise nickel-cobalt-chromium. In some examples, the frame 302 comprises a nickel-cobalt-chromium-molybdenum alloy, such as MP35N™ (tradename of SPS Technologies), which is equivalent to UNS R30035 (covered by ASTM F562-02). MP35N™/UNS R30035 comprises 35% nickel, 35% cobalt, 20% chromium, and 10% molybdenum, by weight.

In some examples, the prosthetic valve 62 or 300 can be a self-expandable prosthetic valve with a frame made from a self-expanding material, such as Nitinol. When the prosthetic valve is a self-expanding valve, the balloon of the delivery apparatus can be replaced with a sheath or similar restraining device that retains the prosthetic valve in a radially compressed state for delivery through the body. When the prosthetic valve is at the implantation location, the prosthetic valve can be released from the sheath, and therefore allowed to expand to its functional size. It should be noted that any of the delivery apparatuses disclosed herein can be adapted for use with a self-expanding valve.

Additional details regarding the prosthetic valves described herein and various valve components are described U.S. Patent No. 11,185,406, which is incorporated herein by reference. Additional example prosthetic valves are described in International Patent Application Publication No. WO 2018/222799, U.S. Patent No. 9,155,619, and U.S. Patent Publication No. 2018/0028310, all of which are incorporated herein by reference in their entireties.

### Sterilization

Any of the systems, devices, apparatuses, etc. herein can be sterilized (for example, with heat/thermal, pressure, steam, radiation, and/or chemicals, etc.) to ensure they are safe for use with patients, and any of the methods herein can include sterilization of the associated system, device, apparatus, etc. as one of the steps of the method. Examples of heat/thermal sterilization include steam sterilization and autoclaving. Examples of radiation for use in sterilization include, without limitation, gamma radiation, ultra-violet radiation, and electron beam. Examples of chemicals for use in sterilization include, without limitation, ethylene oxide, hydrogen peroxide, peracetic acid, formaldehyde, and glutaraldehyde. Sterilization with hydrogen peroxide may be accomplished using hydrogen peroxide plasma, for example.

### Additional Examples of the Disclosed Technology

In view of the above-described implementations of the disclosed subject matter, this application discloses the additional examples enumerated below. It should be noted that one feature of an example in isolation or more than one feature of the example taken in combination and, optionally, in combination with one or more features of one or more further examples are further examples also falling within the disclosure of this application.

Example 1. A docking device for securing a prosthetic valve at a native valve, the docking device comprising: a coil comprising a plurality of helical turns when deployed at the native valve; and a guard member attached to the coil, wherein the guard member comprises braided wires and is movable between a radially compressed state and a radially expanded state, wherein the guard member comprises a plurality of body portions and one or more neck portions connecting the plurality of body portions, wherein when the guard member is in the radially expanded state, a diameter of the neck portions is smaller than a diameter of the body portions.

Example 2. The docking device of any example herein, particularly example 1, wherein the braided wires comprise a shape memory alloy.

Example 3. The docking device of any example herein, particularly any one of examples 1-2, wherein when the guard member is in the radially compressed state, the neck portions and the body portions have a same compressed diameter.

Example 4. The docking device of any example herein, particularly example 3, wherein the compressed diameter is between 1 mm and 4 mm, inclusive.

Example 5. The docking device of any example herein, particularly example 4, wherein the compressed diameter is between 2 mm and 3 mm, inclusive.

Example 6. The docking device of any example herein, particularly any one of examples 1-5, wherein when the guard member is in the radially expanded state, the diameter of the neck portions is between 5 mm and 6.5 mm, and the diameter of the body portions is between 7 mm and 10 mm, all inclusive.

Example 7. The docking device of any example herein, particularly any one of examples 1-5, wherein when the guard member is in the radially expanded state, the diameter of the neck portions and the diameter of the body portions has a diameter ratio between 0.5 and 0.95, inclusive.

Example 8. The docking device of any example herein, particularly example 7, wherein the diameter ratio is between 0.75 and 0.9, inclusive.

Example 9. The docking device of any example herein, particularly any one of examples 1-8, wherein when the guard member is in the radially expanded state, an axial length the neck portions is smaller than an axial length of the body portions.

Example 10. The docking device of any example herein, particularly example 9, wherein when the guard member is in the radially expanded state, the axial length of the neck portions is between 1.5 mm and 2.5 mm, and the axial length of the body portions is between 8 mm and 10 mm, all inclusive.

Example 11. The docking device of any example herein, particularly any one of examples 9-10, wherein when the guard member is in the radially expanded state, the axial length of the neck portions and the axial length of the body portions has a length ratio between 0.1 and 0.3, inclusive.

Example 12. The docking device of any example herein, particularly example 11, wherein the length ratio is between 0.2 and 0.25, inclusive.

Example 13. The docking device of any example herein, particularly any one of examples 1-12, wherein the diameter of the body portion in the expanded state and the diameter of the body portion in the compressed state define a first compression ratio, wherein the first compression ratio is between 2 and 6.

Example 14. The docking device of any example herein, particularly example 13, wherein the diameter of the neck portion in the expanded state and the diameter of the neck portion in the compressed state define a second compression ratio, wherein the second compression ratio is between 1.5 and 4.

Example 15. The docking device of any example herein, particularly any one of examples 1-14, wherein the number of body portions is between two and ten, inclusive.

Example 16. The docking device of any example herein, particularly example 15, wherein the number of body portions is between four and six, inclusive.

Example 17. The docking device of any example herein, particularly any one of examples 1-16, wherein a distal end of the guard member is fixedly attached to the coil, wherein a proximal end of the guard member is axially movable relative to the coil.

Example 18. The docking device of any example herein, particularly any one of examples 1-17, further comprising an outer cover surrounding an outer surface of the guard member.

Example 19. The docking device of any example herein, particularly any one of examples 1-18, further comprising an inner cover fixedly attached to an outer surface of the coil.

Example 20. The docking device of any example herein, particularly example 19, further comprising a retention element surrounding at least a portion of the inner cover, wherein the coil, the inner cover, and the retention element extend through a lumen of the guard member.

Example 21. A docking device for securing a prosthetic valve at a native valve, the docking device comprising: a coil comprising a plurality of helical turns when deployed at the native valve; and a guard member attached to the coil, wherein the guard member is movable between a radially compressed state and a radially expanded state, wherein the guard member comprises a plurality of body portions and one or more neck portions connecting the plurality of body portions, wherein when the guard member is in the radially compressed state, both the body portions and neck portions are reduced to a compressed diameter, wherein when the guard member is in the radially expanded state, the neck portions are radially expanded to a first expanded diameter that is larger than the compressed diameter, wherein the body portions are radially expanded to a second expanded diameter that is larger than the first expanded diameter.

Example 22. The docking device of any example herein, particularly example 21, wherein the guard member comprises a braided wire frame.

Example 23. The docking device of any example herein, particularly example 22, wherein the braided wire frame comprises a shape memory alloy.

Example 24. The docking device of any example herein, particularly any one of examples 21-23, wherein when the guard member is in the radially compressed state, no void space is formed between the guard member and the coil.

Example 25. The docking device of any example herein, particularly any one of examples 21-24, wherein a first end of the guard member is fixedly attached to the coil, wherein a second end of the guard member is axially movable relative to the coil.

Example 26. The docking device of any example herein, particularly example 25, wherein the first end is distal to the second end.

Example 27. The docking device of any example herein, particularly any one of examples 21-26, further comprising an outer cover surrounding an outer surface of the guard member.

Example 28. The docking device of any example herein, particularly any one of examples 21-27, further comprising an inner cover surrounding the coil, wherein a portion of the inner cover is surrounded by the guard member.

Example 29. The docking device of any example herein, particularly example 28, wherein radially expand the prosthetic valve within a space defined by the helical turns of the coil causes the guard member to move from the radially expanded state to a semi-expanded state, wherein at the semi-expanded state, an inner side of at least a portion of the guard member is radially compressed by the prosthetic valve such that inner cover contacts the inner side of the portion of the guard member, while an outer side of the portion of the guard member is radially spaced away from the inner cover.

Example 30. The docking device of any example herein, particularly any one of examples 21-29, wherein each of the neck portions is sized to receive a medial commissure of a native mitral valve.

Example 31. A guard member for a docking device configured to receive a prosthetic valve, the guard member comprising a braided wire frame such that the guard member is movable between a radially compressed state and a radially expanded state, wherein the guard member comprises a plurality of body portions and one or more neck portions connecting the plurality of body portions, wherein when the guard member is in the radially compressed state, both the body portions and neck portions are reduced to a compressed diameter, wherein when the guard member is in the radially expanded state, the neck portions are radially expanded to a first expanded diameter that is larger than the compressed diameter, wherein the body portions are radially expanded to a second expanded diameter that is larger than the first expanded diameter.

Example 32. The guard member of any example herein, particularly example 31, wherein the braided wire frame comprises a nickel titanium alloy.

Example 33. The guard member of any example herein, particularly any one of examples 31-32, further comprising an outer cover surrounding an outer surface of the braided wire frame.

Example 34. The guard member of any example herein, particularly example 33, wherein the outer cover comprises a fabric material.

Example 35. A method comprising: deploying a docking device at an annulus of a native valve; and deploying a prosthetic valve within the docking device, wherein the docking device comprises a coil and a guard member attached to the coil, the coil having a helical configuration after being deployed, wherein the guard member comprises braided wires and is movable between a radially compressed state and a radially expanded state, wherein when the guard member is in the radially expanded state, a diameter of the neck portions is smaller than a diameter of the body portions, wherein deploying the docking device comprises moving the guard member from the radially compressed state to the radially expanded state, and aligning one of the neck portions of the guard member to a commissure of the native valve such that the commissure is positioned between two adjacent body portions of the guard member.

Example 36. The method of any example herein, particularly example 35, wherein the native valve is a mitral valve, and the commissure is a medial commissure of the mitral valve.

Example 37. The method of any example herein, particularly any one of examples 35-36, wherein the two body portions of the guard member located on both sides of the commissure are configured to reduce paravalvular leakage between the prosthetic valve and the native valve at the commissure.

Example 38. The method of any example herein, particularly any one of examples 35-37, further comprising delivering the docking device in a substantially straight configuration to the native valve, wherein delivering the docking device comprises retaining the guard member in the radially compressed state under a dock sleeve, wherein moving the guard member from the radially compressed state to the radially expanded state comprises retracting the dock sleeve so as to expose the guard member.

Example 39. The method of any example herein, particularly any one of examples 35-38, wherein deploying the prosthetic valve comprises radially expanding the prosthetic valve so as to cause the guard member to move from the radially expanded state to a semi-expanded state, wherein at the semi-expanded state, an inner side of at least a portion of the guard member is radially compressed by the prosthetic valve so that the inner side of the portion of the guard member moves closer to the coil.

Example 40. A method for making a guard member for a docking device configured to receive a prosthetic valve, the method comprising: braiding a layer of metal wires over a mandrel to form an initial state of the guard member, wherein the metal wires comprise a shape memory material, wherein the mandrel comprises a plurality of body portions and one or more neck portions connecting the plurality of body portions, wherein a diameter of the neck portions is smaller than a diameter of the body portions; radially compressing portions of metal wires over the neck portions of the mandrel to transform the guard member from the initial state to a temporarily deformed state, wherein the guard member in the temporarily deformed state conforms to a shape of the mandrel; heating the guard member at a predetermined temperature over a predetermined duration to transform the guard member from the temporarily deformed state to a permanently deformed state, wherein the heating shape sets the layer of metal wires so that the guard member in the permanently deformed state retains the shape of the mandrel when no external force is applied to the guard member.

Example 41. A method comprising sterilizing the docking device or guard member of any example herein, particularly any one of examples 1-34.

Example 42. A method of treating a heart on a simulation, the method comprising: deploying a docking device at a target location; and deploying a prosthetic valve within the docking device; wherein the docking device is according to any one of examples 1-30.

The features described herein with regard to any example can be combined with other features described in any one or more of the other examples, unless otherwise stated. For example, any one or more of the features of one docking device can be combined with any one or more features of another docking device. As another example, any one or more features of one guard member can be combined with any one or more features of another guard member.

In view of the many possible examples to which the principles of the disclosed technology may be applied, it should be recognized that the illustrated examples are only preferred examples of the technology and should not be taken as limiting the scope of the disclosure. Rather, the scope of the claimed subject matter is defined by the following claims and their equivalents.

What is claimed is:

1. A docking device for securing a prosthetic valve at a native valve, the docking device comprising:
a coil comprising a plurality of helical turns when deployed at the native valve; and
a guard member attached to the coil,
wherein the guard member comprises braided wires and is movable between a radially compressed state and a radially expanded state,
wherein the guard member comprises a plurality of body portions and one or more neck portions connecting the plurality of body portions,
wherein when the guard member is in the radially expanded state, a diameter of the neck portions is smaller than a diameter of the body portions.

2. The docking device of claim 1, wherein the braided wires comprise a shape memory alloy.

3. The docking device of claim 1, wherein when the guard member is in the radially compressed state, the neck portions and the body portions have a same compressed diameter.

4. The docking device of claim 1, wherein when the guard member is in the radially expanded state, an axial length the neck portions is smaller than an axial length of the body portions.

5. The docking device of claim 1, wherein the number of body portions is between two and ten, inclusive.

6. The docking device of claim 1, wherein a distal end of the guard member is fixedly attached to the coil, wherein a proximal end of the guard member is axially movable relative to the coil.

7. The docking device of claim 1, further comprising an outer cover surrounding an outer surface of the guard member.

8. The docking device of claim 1, further comprising an inner cover fixedly attached to an outer surface of the coil.

9. The docking device of claim 8, further comprising a retention element surrounding at least a portion of the inner cover, wherein the coil, the inner cover, and the retention element extend through a lumen of the guard member.

10. A docking device for securing a prosthetic valve at a native valve, the docking device comprising:
a coil comprising a plurality of helical turns when deployed at the native valve; and
a guard member attached to the coil,
wherein the guard member is movable between a radially compressed state and a radially expanded state,
wherein the guard member comprises a plurality of body portions and one or more neck portions connecting the plurality of body portions,
wherein when the guard member is in the radially compressed state, both the body portions and neck portions are reduced to a compressed diameter,
wherein when the guard member is in the radially expanded state, the neck portions are radially expanded to a first expanded diameter that is larger than the compressed diameter, wherein the body portions are radially expanded to a second expanded diameter that is larger than the first expanded diameter.

11. The docking device of claim 10, wherein the guard member comprises a braided wire frame.

12. The docking device of claim 11, wherein the braided wire frame comprises a shape memory alloy.

13. The docking device of claim 10, wherein when the guard member is in the radially compressed state, no void space is formed between the guard member and the coil.

14. The docking device of claim 10, wherein a first end of the guard member is fixedly attached to the coil, wherein a second end of the guard member is axially movable relative to the coil.

15. The docking device of claim 14, wherein the first end is distal to the second end.

16. The docking device of claim 10, further comprising an outer cover surrounding an outer surface of the guard member.

17. The docking device of claim 10, further comprising an inner cover surrounding the coil, wherein a portion of the inner cover is surrounded by the guard member.

18. The docking device of claim 17, wherein radially expand the prosthetic valve within a space defined by the helical turns of the coil causes the guard member to move from the radially expanded state to a semi-expanded state, wherein at the semi-expanded state, an inner side of at least a portion of the guard member is radially compressed by the prosthetic valve such that inner cover contacts the inner side of the portion of the guard member, while an outer side of the portion of the guard member is radially spaced away from the inner cover.

19. The docking device of claim 10, wherein each of the neck portions is sized to receive a medial commissure of a native mitral valve.

20. A method comprising:
deploying a docking device at an annulus of a native valve; and
deploying a prosthetic valve within the docking device,
wherein the docking device comprises a coil and a guard member attached to the coil, the coil having a helical configuration after being deployed,
wherein the guard member comprises braided wires and is movable between a radially compressed state and a radially expanded state,
wherein when the guard member is in the radially expanded state, a diameter of the neck portions is smaller than a diameter of the body portions,
wherein deploying the docking device comprises moving the guard member from the radially compressed state to the radially expanded state, and aligning one of the neck portions of the guard member to a commissure of the native valve such that the commissure is positioned between two adjacent body portions of the guard member.
